(19) United States
(12) Patent Application Publication
(10) Pub. No.: US 2015/0148446 A1
(43) Pub. Date: May 28, 2015

(54) Use of an Unfilled or Filler-Filled, Organically-Modified Silicic Acid (Hetero)Polycondensate in Medical and Non-Medical Processes for Modifying the Surface of a Body Comprised of a Previously Hardened, Unfilled or Filler-Filled Silicic Acid (Hetero) Polycondensate in Particular for Dental “Chairside” Treatment

(71) Applicant: Fraunhofer-Gesellschaft Zur Foerderung der angewandten Forschung e.V., Muenchen (DE)

(72) Inventors: Herbert Wolter, Tauberbischofsheim (DE); Florian Hausler, Wuerzburg (DE)

(21) Appl. No.: 14/399,833

(22) PCT Filed: May 3, 2013
(86) PCT No.: PCT/EP2013/059248

§ 371 Date: Nov. 7, 2014

(30) Foreign Application Priority Data
May 11, 2012 (DE) 102012104139.3

(51) Int. Cl. A61K 6/083

(52) U.S. Cl. CPC A61K 6/083
USPC 523/118

(57) ABSTRACT

The present invention relates to an organically-modified, organically cross-linked silicic acid (hetero) polycondensate or composite, comprising an organically-modified, organically cross-linked silicic acid (hetero) polycondensate and a filler, for intraoral application, particularly for application when repairing an existing dental replacement in the mouth of a patient or for applying an enamel layer to an anatomically-reduced, milled crown base body already located in the mouth of a patient, wherein said existing dental replacement or said crown base body is comprised of a material having free hydroxy, carboxyl, thio or amino groups, or forming these on its surface following a physical-chemical treatment and/or potentially having active C═C double bonds, comprising the following steps in the mouth of the patient:
(a) If necessary, processing of the previously existing dental replacement to remove damaged or no longer necessary elements,
(b) Applying a bonding agent to the surface of said potentially processed dental replacement or said crown base body,
(c) Overlaying the surface of said potentially processed dental replacement or said crown base body coated with bonding agent with said organically-modified silicic acid (hetero) polycondensate or said composite in a liquid or paste-like form, and
(d) Hardening the overlay achieved pursuant to (c) through organic cross-linking by means of light hardening or redox-induced hardening,
   wherein said bonding agent is or has a compound having at least two identical substituents selected from among hydroxy groups, isocyanate groups, epoxy groups, potentially activated carboxylic acid groups, thiol groups, primary and secondary amino groups of cyclical carbonate groups having a potentially activated C═C double bond.

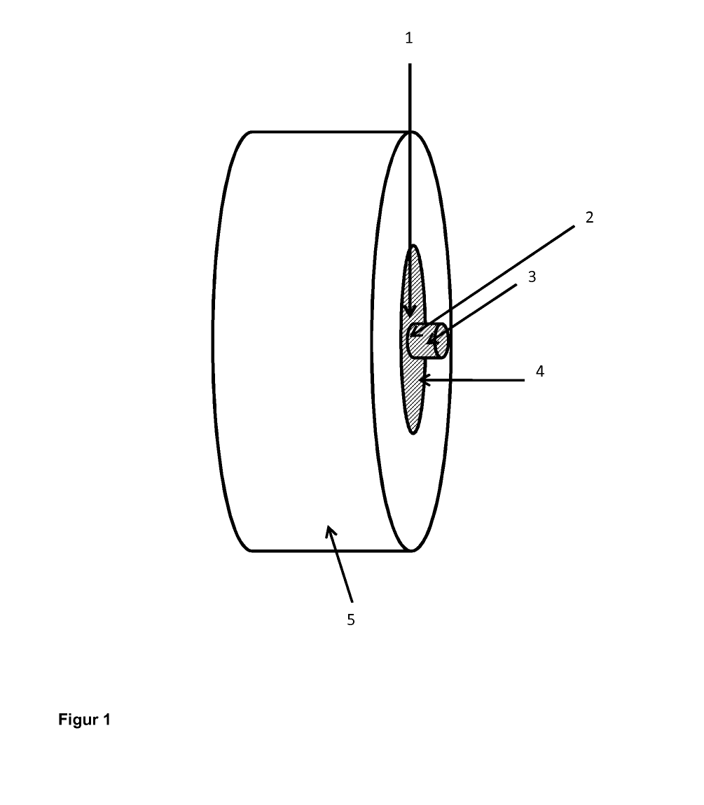

The invention further relates to the use of the specified materials for dental purposes conducted in a laboratory, as well as for purposes beyond the field of dentistry.

The present invention relates to the alteration of a surface of a previously hardened molded article comprised of an unfilled or filler-filled, organically-modified silicic acid (hetero) polycondensate with the aid of an additional silicic acid (hetero) polycondensate material, in particular when repairing a dental replacement or for finishing crowns based on organically-modified silicic acid (hetero) polycondensates in a dental laboratory or within the scope of “chairside” treatment, wherein an organically-modified silicic acid (hetero) polycondensate is used as a repair or incisal material as well as potentially a bonding agent. Throughout the course of the process used in this case, the surface of said previously hardened molded article, e.g. an “old” tooth replacement, is potentially roughened and then coated with a bonding agent. Said bonding agent has a strong adhesion to said dental replacement comprised of filled or unfilled, organically-modified, normally organically cross-linked silicic acid (hetero) polycondensates, and thus enables a particularly good bond of this dental replacement to an in turn hardened repair or incisal material.

A composite-based direct or indirect dental replacement is used quite frequently in modern dentistry. However, these materials are exposed to sever mechanical and chemical stress in the oral environment, as a result of which defects, such as fractures, wear caused by chewing (abrasion), etc. may occur. To remedy these defects to the tooth replacement, it is still very often completely replaced today. However, this always leads to a portion of healthy tooth substance being removed during this replacement. There are two reasons for this. The transition to hard dental substance is difficult to identify due to the tooth-colored composite; grinding the dental replacement down exactly is still complicated due to the strong bond of the materials to the dental replacement achieved by modern adhesives. A replacement of a restoration is also frequently accompanied by renewed local anesthesia, which additionally strains the body of the patient. To repair this tooth replacement, a force-fit bond from the old dental replacement to the subsequently applied repair material would have to be achieved.

One specific problem can be seen with respect to a multi-layered structure of composite crowns, as is described, for example, in EP 2 246 008 A2 and in DE 10 2012 202 005.5, which has not yet been published. This multi-layered structure consists of a, e.g. thermally hardened crown base body, which was milled in an anatomically reduced manner via CAD/CAM device. Subsequently, A highly translucent composite (enamel layer=so-called incisal material) is subsequently applied to a hardened base body and, e.g. hardened by means of blue light. If said body was manufactured from or with a silicic acid (hetero) polycondensate, an originally present “smear layer” on the surface of said body, which is relatively soft and sticky and still bears several reactive groups of the output material, is lost due to the hardening and subsequent finishing of the base body (e.g. grinding). This layer is also referred to as an oxygen inhibition layer because its formation actually has to do with the fact that complete hardening on the surface is inhibited through oxygen; however, its exact genesis is not known. It substantially contributes to a good adhesion of the body coated with it; if it is ground away or lost due to blue light hardening, achieving an optimum bond between said hardened and subsequently applied composite (likewise when repairing a composite-based tooth replacement) is extremely difficult.

The cost pressure on the healthcare system can be countered with cost-efficient, but still high-quality dental replacement systems. The materials suggested for this in DE 10 2012 202 005.5, which had not yet been published on the priority date of the present application, based on organically-modified silicic acid (hetero) polycondensates for use as crowns, inlays, onlays (3-unit bridges), repair systems for crowns, basic prosthesis materials, dentures, and veneer materials for the so-called “chairside” application (for application by the dentist or his staff directly in the dentist's chair, i.e. during or as a part of the treatment) are specified below:
(a) Chairside crowns, inlays, onlays (3-unit bridges), temporary crowns:
   The molding can be produced as blocks, e.g. as disc-shaped circular blocks, from which multiple crowns can be milled, (multi-layered design potentially made from variously colored materials, the translucency of which preferably increases from the inside outward, wherein each layer is separately pre-hardened and the entire block is then re-hardened, or one color—then compact design), or as highly-filled monochrome, opaque blocks, which are additionally covered in specific embodiments with a pasty, translucent incisal material, which is applied, hardened, and subsequently re-milled. Said prefabricated blocks can be additionally individualized with the help of a light-hardened paint set. Inexpensive fillers can be used for temporary care, e.g. of the mentioned crowns.
(b) Basic prosthesis materials, dentures, veneer material:
   These materials should be translucent, have a high breaking strength, and be easy to process. For example, norbornene-based matrices (output materials, e.g. silanes of a general formula F—see below) are suitable for this. Shrinkage should be as minimal as possible to ensure the form stability, which can be realized through the incorporation of fillers. For instance, fillers adapted to the refractive index as well as pearl and/or splinter polymerisates are possible. The matrix should be capable of being hardened in a redox-induced manner; mixed material should be capable of being cast to the extent possible. However, thermal hardening is possible as well; due to the individuality of prostheses and the resulting different material thicknesses, light-hardening may however be precluded.

The specified bodies and materials are manufactured from or with optionally filled silicic acid (hetero) polycondensates, particularly as a repair material, basic prosthesis material, and veneer material, as well as from filled, preferably thermally cross-linked silicic acid (hetero) polycondensates for moldings for “chairside” crowns, inlays and onlays, and dentures, frequently in a surgical or therapeutic procedure to be conducted by a dentist.

The silicic acid (hetero) polycondensates capable of being used represent a common material basis for all aforementioned materials. Thus, they can be combined better, wherein properties such as esthetics, impact strength, breaking strength, modulus of elasticity, abrasion, and the like can be adjusted according to the individual indication for a precise composition of the selected resin matrix, the type of filler, and their shares to each other.

The silicic acid (hetero) polycondensates capable of being used have a radical in common, which is bonded to silicon via carbon and normally bears at least one organically polymerizable group or one reactive ring. An organically polymerizable group presently means that this group is accessible to a polyreaction, for which reactive double bonds or rings transform into polymers (addition polymerization or chain-growth polymerization) under the influence of heat, light, ionizing radiation or redox-induced (e.g. with an initiator (peroxide or the like) and an activator (amine or the like)). During this polymerization, neither a separation of molecular components occurs nor a migration or rearrangement. Moreover, these groups should particularly preferably be accessible to a thiol-ene polyaddition when a thiol is added; even primary and secondary amines (particularly with at least two, though even three, four or more amino groups) should be able to be deposited. Alternatively, they can be accessible to a ROMP (ring opening metathesis polymerization). Examples for this are norbornene groups. The reactive double bond(s) of this group can be randomly selected, for example, a vinyl group or component of an allyl or styryl group. Preferably, it/they are a component of a double bond, which is accessible to a Michael addition, thus containing an activated methylene group as a result of the proximity to a carbonyl group. In turn, preferred among these are acrylic acid and methacrylic acid groups or derivatives. The organically polymerizable group normally contains at least two and preferably up to approx. 100 carbon atoms. It can be bonded to the carbon network of the Si—C bonded radical directly or via a random linkage group.

The term “(meth)acrylic . . . ” presently means that in each case it can be dealing with the respective acrylic or the respective methacrylic compound or a mixture of both. The present (meth)acrylic acid derivatives comprise the acids themselves, potentially in an activated form—esters, amides, thioesters, and the like.

The organically modified silicic acid polycondensates in DE 10 2012 202 005.5 may be exclusively silicon-based; however, instead they may have additional metal atoms in the inorganic framework as well, such as is known from the state of the art. These will be designated at present as silicic acid hetero polycondensates. The term “silicic acid (hetero) polycondensates” should comprise both variations. The condensates contain organic radicals bonded to silicon via carbon.

Examples for silicic acid hetero polycondensates usable pursuant to the invention, which are by no means limited, can be produced from the following silanes.

Silanes of a general formula (A):

{XₐR_{b}Si[R(A)_{c}]_{(4-a-b)}}ₓB (A)

wherein said radicals have the following meaning:
X: hydroxy, alkoxy, acyloxy, alkylcarbonyl, alkoxycarbonyl or —NR″2;
R: alkyl, alkenyl, aryl, alkylaryl or arylalkyl;
R′: alkylene, arylene or alkylenarylene;
R″: hydrogen, alkyl or aryl;

### A: 0, S, PR″, POR″ or NHC(O)O;

B: straight-chain or branched out organic radical that is derived from a compound with at least two C═C double bonds and 5 to 50 carbon atoms;
a: 1, 2 or 3;
b: 0, 1 or 2;
c: 0 or 1;
x: whole number, the maximum value of which corresponds to the number of double bonds in the compound B minus 1,

Such silanes and polycondensates produced therewith are revealed in DE 40 11 044 A.

Silanes of a general formula (B):

B{A-(Z)_{d}—R¹(R²)—R′—SiXₐR_{b}}_{c} (B)

wherein said radicals and indices have the following meaning:

### A=O, S, NH or C(O)O;

B=straight-chain or branched out organic radical with at least one C═C double bond and 4 to 50 carbon atoms;
R=alkyl, alkenyl, aryl, alkylaryl or arylalkyl;
R′=alkylene, arylene, arylenalkylene or alkylenarylene with respectively 0 to 10 carbon atoms, whereas these radicals can be interrupted by oxygen and sulfur atoms or by amino groups;
R¹=nitrogen, alkylene, arylene or alkylenarylene with respectively 1 to 10 carbon atoms, wherein these radicals can be interrupted by oxygen or sulfur atoms or by amino groups;

### R²=H, OH or COOH;

X=hydrogen, hydroxy, alkoxy, acyloxy, alkylcarbonyl, alkoxycarbonyl or —NR″₂;
R″=alkyl or aryl;
Z=CO or CHR, with R equal to H, alkyl, aryl or alkylaryl;
a=1, 2 or 3;
b=0, 1 or 2;
c=1, 2 or 3
d=0 or 1

Such silanes and silicic acid polycondensates produced therewith are revealed in DE 44 16 857 C1.

Silanes of a general formula (C)

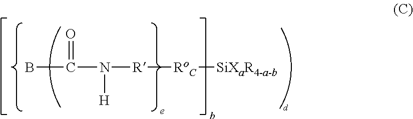

wherein said radicals and indices have the following meaning:

B=organic radical with at least one C═C double bond;

R=alkyl, alkenyl, aryl, alkylaryl or arylalkyl;
R^{o} and R′ respectively=alkylene, alkenylene, arylene, alkylenarylene or arylenalkylene;
X=hydroxy, alkoxy, acyloxy, alkylcarbonyl, alkoxycarbonyl or —NR″₂ with R″ equal to hydrogen, alkyl or aryl;
a=1, 2 or 3
b=1, 2 or 3, with a+b=2, 3 or 4;
c=0 or 1;
d=1, 2, 3, 4, 5, 6, 7, 8, 9 or 10;
e=1, 2 or 3 or 4; with e equal to 1 for c=0.

Said silanes of formula (C) and silicic acid polycondensates capable of being derived thereof are revealed in DE 199 10 895 A1. Silanes of a general formula (D):

{B′-Z′-R¹(B)-R-}ₐ(R′)_{b}SiX_{4-a-b} (D)

wherein said radicals and indices have the following meaning:
R is an alkylene, arylene or alkylenarylene group, which can be interrupted by one or more oxygen or sulfur atoms or carboxyl or amino groups, or can carry such atoms/groups on its end facing away from the silicon atom;
R¹ is an alkylene, arylene or alkylenarylene group substituted by Z′, which can be interrupted by one or more oxygen or sulfur atoms or carboxyl or amino groups, or can carry such atoms/groups on one of its ends;
R′ is an alkyl, alkenyl, aryl, alkylaryl or arylalkyl group;
B and B′ can be equal or different; both radicals have the meaning of a straight-chain or branched organic group with at least one C═C double bond and at least two carbon atoms;
X is a group, which can enter a hydrolytic condensation reaction through the formation of Si—O—Si bridges (with the exception of hydrogen and halogen);
Z′ have the meaning —NH—C(O)O—, —NH—C(O)— or —CO(O)—, wherein both of the initially mentioned radicals are bonded to radical B′ by an NH group, while a carboxylate group can point in both directions;
a represents 1 or 2 and
b is 0 or 1;

Such silanes and polycondensates capable of being derived thereof are revealed in DE 103 49 766 A1.

Silanes of a general formula (E):

(XₐR_{b}Si)ₘ[-{B}-([O]ₒP[O]ₚR′^{c}Y_{d})ₙ]_{4ab} (E)

wherein said groups, radicals, and indices have the following meaning:
B is at least a double-bonded, straight-chain or branched group with at least one organically polymerizable radical and at least 3 carbon atoms,
X is a radical or OH capable of being hydrolyzed off a silicon atom (with the exception of hydrogen and halogen),
R and R′ are independent and potentially substituted alkyl, alkenyl, aryl, alkylaryl or arylalkyl,

### Y is OH or OR′,

a is 0, 1, 2 or 3,
b is 0, 1 or 2, whereas a+b together are 1, 2 or 3,
c is 0, 1 or 2,
d is 0, 1 or 2,
c+d together are 2,
m is at least 1, with the stipulation that m is not greater than 1 if a+b represents 1 or 2,
n is at least 1,
o is 0 or 1, and
p is 0 or 1,

Silanes of formula (E) and silicic acid polycondensates derived thereof are revealed in DE 101 32 654 A1.

Silanes of a general formula (F):

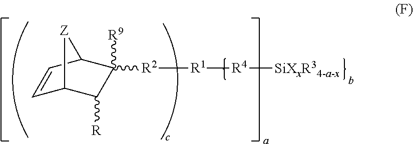

wherein said radicals and indices are equal or different and have the following meaning: R is hydrogen, R²—R¹—R⁴—SiXₓR³₃₋ₓ, carboxyl, alkyl, alkenyl, aryl, alkylaryl or arylalkyl,
R¹ and R² are independent alkylene, arylene, arylenalkylene or arylenalkylene,
R³ is alkyl, alkenyl, aryl, alkylaryl or arylalkyl,
R⁴ is —(CHR⁶—CHR⁶)ₙ— with n=0 or 1, CHR⁶—CHR⁶—S—R⁵—, —C(O)—S—R⁵—, CHR⁶—CHR⁶—NR⁶—R⁵, —Y—C(S)—NH—R⁵, —S—R⁵, —Y—C(O)—NH—R⁵—, —C(O)—O—R⁵—, —Y—CO—C₂H₃(COOH)—R⁵—, —Y—CO—C₂H₃(OH)—R⁵— or —C(O)—NR⁶—R⁵,
R⁵ is alkylene, arylene, arylenalkylene or arylenalkylene,
R⁶ is hydrogen, alkyl or aryl with 1 to 10 carbon atoms,
R⁹ is hydrogen, alkyl, alkenyl, aryl, alkylaryl or arylalkyl,
X is hydroxy, alkoxy, acyloxy, alkylcarbonyl or alkoxycarbonyl;

### Y is —O—, —S— or NR⁶,

Z is —O— or —(CHR⁶)ₘ with m equal to 1 or 2;
a is 1, 2 or 3, with b=1 for a=2 or 3
   b is 1, 2 or 3, with a=1 for b=2 or 3
   c is a whole number from 1 to 6,
   x is 1, 2 or 3 and
   a+x are 2, 3 or 4.

These silanes and silicic acid polycondensates derived thereof are revealed in DE 196 27 198 A1.

Additional silicic acid (hetero) polycondensates usable pursuant to the invention comprise (meth)acrylic radicals and either sulfonate or sulfate groups that are respectively bonded directly or indirectly to a silicon atom via a non-substituted or substituted hydrocarbon group with a C—Si bond. These condensates can be produced, for example, from silanes of a formula (G)

R¹ₐR²_{b}SiZ_{4-a-b} (G)

wherein R¹ is a hydrolytically condensable radical, R² is substituted or non-substituted, straight-chained, branched or a cycle having alkyl, aryl, arylalkyl, alkylaryl or alkyl/arylalkyl or is a respective alkenyl, a carbon chain of may be interrupted in any event potentially by —O—, —S—, —NH—, —S(O)—, —C(O)NH—, —NHC(O)—, —C(O)O— —C(O)S, —NHC(O)NH— or C(O)NHC(O) groups, which may potentially point in both possible directions, Z is a radical, in which at least one (meth)acrylic group and at least either one sulfonate or one sulfate group is bonded directly or indirectly to a silicon atom via a non-substituted or substituted hydrocarbon group with a C—Si bond, a is 1, 2 or 3, b is 0, 1 or 2, and a+b together are 2 or 3. Specific silanes of said formula (G) have a following formula (G′)

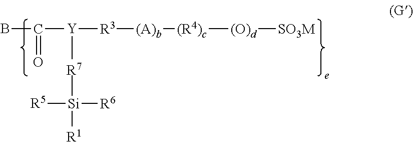

wherein
R³ is an alkylene, which is non-substituted or substituted with a functional group, straight-chained, branched, or having at least one cycle,
A represents a linkage group,
R⁴ represents an alkylene, which is potentially interrupted by O, S, NH or NR⁸ and/or potentially functionally substituted,
M is hydrogen or a monovalent metal cation or a respective share of a polyvalent metal cation, preferably selected from Na, K, ½Ca, ½Mg and ammonium,
R⁵ and R⁶ independently have either the meaning of R¹ or are alkyl, aryl, arylalkyl, alkylaryl or alkyl/arylalkyl, which are substituted or non-substituted, straight-chained, branched, or having at least one cycle; as an exception, however, may instead be a respective alkylene, arylalkylene or alkylenaryl,
R⁷ is a hydrocarbon group bonded to a silicon atom via a carbon atom, as was described further above,
R⁸ is C₁-C₆-alkyl or (meth)acrylic,
B is vinyl, 2-allyl or, in the case of e>1, an organic radical having e vinyl groups, which are respectively bonded to a group within the curly brackets
Y is a nitrogen atom, —O—CH═, —S—CH═ or —NH—CH═, whereas said oxygen atom, sulfur atom or said NH group has a bond to the neighboring C(O) group,
b=0 or 1
c=0 or 1
with the stipulation that, for the combination of Y equal to a nitrogen atom, b=0 and c=0 of said radical R³, as well as the stipulation that, for the combination of Y equal to a nitrogen atom, b=0 and c=1 of said radical R⁴ represents an alkylene interrupted by O, S, NH or NR⁸, and which is potentially functionally substituted,
d=0 or 1, and
e=1, 2 or 3.

Such materials are revealed in application DE 10 2011 050 672.1, which has likewise not yet been published.

Additional silicic acid (hetero) polycondensates capable of being used can be produced through a process, wherein a silane or siloxane having a radical bonded via a carbon atom to a silicon atom, which bears at least two functional groups, whereas a first functional group of the two is an unsaturated, organically polymerizable group and a second functional group of the two is selected from (a) additional unsaturated, organically polymerizable groups, (b) COOR⁸ or —(O)_{b}P(O)(R⁵)₂ and (c) —OH, with R⁸ equal to R⁴ or M_{1/x}^{x+}, whereas M^{x+} is hydrogen or an x-fold positively charged metal cation, and b=0 or 1, is converted with a compound of a formula (H)

X-W-(Z)ₐ (H)

wherein X is SH, NH₂ or NHR⁴, Z is OH, a carboxylic acid radical —COOH or a salt or an ester of this radical or a silyl radical, W is a substituted or non-substituted hydrocarbon radical, the chain of which can be interrupted by —S—, —O—, —NH—, —NR⁴—, —C(O)O—, —NHC(O)—, —C(O)NH—, —NHC(O)O—, —C(O)NHC(O)—, —NHC(O)NH—, —S(O)—, —C(S)O—, —C(S)NH—, —NHC(S)—, —NHC(S)O—, and a represents 1, 2, 3, 4 or a greater whole number, wherein R⁴ is a non-substituted or substituted hydrocarbon radical or OR⁶, R⁶ is hydrogen or a non-substituted or substituted hydrocarbon radical. The product of the first reaction is then converted in a second reaction with a compound (J)

Y-(W)k-(R1)b (J)

wherein Y is NCO, epoxy or—said radical or radicals Z is (a) hydroxy group(s) in the product of the first reaction—COA′, W is defined for compound (H) as above, R¹ is unsaturated, organically polymerizable group, A′ is hydroxy, a halogenide or —OC(O)R⁴ with R⁴ equal to a non-substituted or substituted hydrocarbon radical, k=0 or 1, whereas k=0 is only possible in the event that Y represents COA′ and b=1 or greater than 1.

This process is described in patent application DE 10 2011 053 865.8, which is unpublished. If, in the process, silanes are used as output materials, a hydrolytic condensation will occur prior to, during or after any of the specified process steps.

The materials in DE 10 2012 202 005.5 involve either masses comprised of organically polymerizable silicic acid (hetero) polycondensates, which, e.g. may be modified to achieve a higher degree of organic cross-linking—potentially with organic compounds or other materials, or composites, i.e. polymerizable silicic acid (hetero) polycondensates potentially modified with organic compounds/materials, which are filled with fillers. The fillers can have any form and be especially particle-like and/or fibrous (particularly short fibers). The fillers, for example, that are described in DE 196 43 781, DE 198 32 965, DE 100 184 05, DE 100 41 038, DE 10 2005 061 965, and DE 10 2005 018 305 are suitable. If necessary, a very high filler content can be achieved.

The condensates are hardened via the above-presented polymerization reaction of groups containing double bonds and/or have rings. As an initiator for the thermal hardening, dibenzoyl peroxide (DBPO) is normally released in the respective Resin System; other suitable hardeners are naturally likewise possible, such as those known from the state of the art. An organic cross-linking may occur through the addition of dimeric or oligomeric organic compounds to respective C═C double bonds, e.g. of thiols or amines with two or more thiol or amino groups. If thiols or amines in the deficit are used with regard to the available double bonds, remaining double bonds can be subsequently hardened with light.

In addition to inorganic-organic hybrid polymers, purely organic dental Resin Systems are naturally used as well. Examples for this are, e.g. Bis-GMA with OH and C═C groups, UDMA and TEGDMA exclusively with C═C groups. However, mixtures of these Resin Systems are possible as well

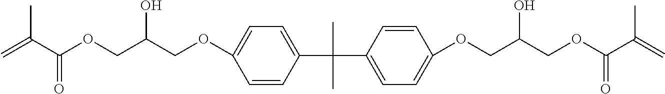 [SMILES: C=C(C)C(=O)OCC(O)COc1ccc(C(C)(C)c2ccc(OCC(O)COC(=O)C(=C)C)cc2)cc1]

Bis-GMA (Bisphenol-A-glycidyl dimethacrylate)

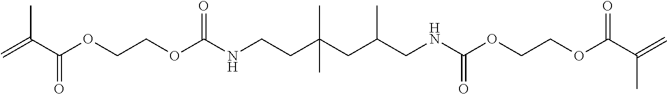 [SMILES: C=C(C)C(=O)OCCOC(=O)NCCC(C)(C)CC(C)CNC(=O)OCCOC(=O)C(=C)C]

UDMA (urethane dimethacrylate)

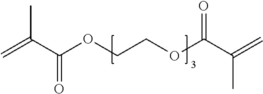

TEGDMA (triethylene glycol dimethacrylate)

Generally, thermal hardening is possible in all cases, in which said material can be hardened prior to being inserted into the mouth of the patient or is not intended for dental purposes. In contrast, hardening reactions that must occur in the mouth of the patient should normally be conducted in a light-induced (e.g. using blue light) or redox-induced manner.

The purpose of the invention is to find a process or a means, with which a previously existing tooth replacement, which was made from or with a hardened silicic acid (hetero) polycondensate, though in a number of cases, from a purely organic material as well, can subsequently be repaired or modified in the mouth of the patient or in the laboratory without affecting the natural tooth material. Said existing dental replacement may be chairside crowns, inlays, onlays, (3-unit bridges), crowns, basic prosthesis materials, dentures, veneer material or fillings. This previously existing dental replacement will also be designated as “old” dental replacement in the following. The term “modify” includes the application of a, for example, highly translucent composite, which is applied to a previously hardened crown base body or the like (in the laboratory or in the mouth of the patient) as an enamel layer or so-called incisal material and then hardened to lend the crown or the like the most natural appearance possible. In specific cases, the purpose of the invention additionally includes the repair or modification of said materials mentioned above for dental purposes as well as in other technical fields.

To meet this challenge, a repair or incisal material is provided in combination with a bonding agent, wherein said repair or incisal material is made from or with a silicic acid (hetero) polycondensate, which may, but does have to be identical thereto, in the (normally occurring) event that the previously existing dental replacement was also made from or with a silicic acid (hetero) polycondensate.

In this regard, the condensate of said repair or incisal material should have groups bonded to carbon atoms selected amongst —CO₂H, —OH, —NHR, —SH and groups with potentially activated C═C double bonds, or groups bonded to silicon selected amongst —OH and —OR, wherein R is respectively selected amongst alkyl, aryl, alkylaryl having preferably 1 to 6 carbon atoms for non-arylized groups and preferably 6-16 carbon atoms for arylized groups.

Said condensate preferably has free hydroxy groups and/or potentially activated carboxylic acid groups and/or groups containing C═C double bonds. If free hydroxy groups or potentially activated carboxylic acid groups are present, the hardening mechanism and, therefore the presence of specific groups that cause the hardening are irrelevant. However, if the hardening of the previously existing dental replacement occurs with the aid of a thiol-ene polyaddition, in some cases, free SH groups can still be present on its surface, for example, due to the use of a surplus of thiol or due to an incomplete reaction. In these cases, a condensate having SH groups bonded to carbon atoms may be selected as a repair or incisal material. In a similar way, the surface of the “old” dental replacement can instead have —NHR groups, e.g. if the source material was made using silanes containing amino groups, the amino groups of which, for example, were only partially used through a reaction with an activated double bond or were not even involved in the hardening reaction. In these cases, a condensate having NHR groups bonded to carbon atoms may be selected as a repair or incisal material.

The selection of said condensate from the group of said aforementioned silicic acid (hetero) polycondensate is particularly preferred. The inventors were able to find a process, with which such a repair or incisal material can be bonded with an old material in a force-fitting, durable, and permanently stabile manner. In doing so, the invention is not limited to dental purposes; thus, the surface of any body from by hardened silicic acid (hetero) polycondensate can be modified/changed according to the process.

For cases, in which a silicic acid (hetero) polycondensate or a composite was used with such a silicic acid (hetero) polycondensate as well as one or more fillers as a dental replacement material for crowns, fillings, and the like, the invention accordingly suggests using the same or similar material (i.e. a material that was likewise made from or with a silicic acid (hetero) polycondensate) as a repair or incisal material for producing multilayer crowns. In the process, the problem depicted previously at the onset, i.e. that the base body no longer has a smear layer (oxygen inhibition layer) through the hardening and the mechanical post-processing required in most cases, and therefore, can only be bonded with the additional material in a force-fitting manner with considerable difficulty. For the chemical activation of the surface, an adhesion promoting layer is applied pursuant to the invention prior to applying said repair or incisal material in order to improve the adhesion between both materials respectively containing a silicic acid (hetero) polycondensate. This layer can be produced by applying at least one difunctional bond, which, on the one hand, makes a permanent bond on the surface of the “old” dental replacement with the reactive groups and, on the other hand, reacts with functional groups of the new silicic acid (hetero) polycondensate in such a manner that a high shear strength is achieved, which ideally meets or exceeds the strength of the “old” dental replacement.

Alternatively to chemical activation through a bonding agent, it is possible to physically enlarge the surface of the “old” material through roughening, e.g. with the help of blasting (“sandblasting”), wherein the particle size is favorably in the range of approx. 11 to 500 μm, preferably in the range of approx. 50-250 μm. This sandblasting can be done at a high pressure (e.g. approx. 1.5 to 4 bar). Alternatively, sandpaper can be used for roughening, e.g. with P500-P1000 grit sandpaper (grit size according to the European FEPA standard). Upon choosing these means, a specialist can be sure that a good bond with desirable properties has been achieved if the shear strength of the material bond (measured with the method of measurement shown below, is so high that the point of failure is not in the bond, but rather in the respective composite containing silicic acid (hetero) polycondensate.

An additional improvement of the bond can be achieved in each of the aforementioned embodiments if the surface of the “old” dental replacement becomes swollen with the help of a solvent. By doing this, the bonding agent can penetrate further or better into the areas of the dental replacement close to the surface, which enlarges the effective surface available for the bond. Moreover, by means of etching, Si—O—Si bridges can be split through the formation of SiOH groups, and organic ester bonds can be split through the formation of respective one hydroxy group and one carboxylic group. Thus, the surface primarily of the “old” dental replacement can be modified through the (re)generation of additional reactive functional groups.

A particularly good bond can be achieved by first physically roughening the surface of the “old” material and/or chemically pre-treating it (e.g. through etching, swelling). The bonding agent and finally the repair or incisal material is applied and hardened to the physically/mechanically and/or chemically-activated surface, which may be done with an application in the mouth (intraoral) most often through photochemical reaction, though incidentally in a redox-induced manner as well. Extraoral applications also enable thermal/IR hardening. Adhesion can be assisted through activation of the respectively involved reactive groups, e.g. through heating or exposure.

Surprisingly, in the process we found that a number of di or even multifunctional compounds cause adhesion of the—then hardened—repair material on the “old” dental replacement, which are significantly superior compared to the repair material applied directly and without bonding agents as well as bonding material known from the state of the art. They are suitable for the bonding agents usable pursuant to the invention. Depending on the repair or incisal material used, the selection of which, as mentioned, is based on the material of the “old” dental replacement, said functional groups of these compounds can be selected according to the following diagram:

| | | active groups on the surface |
|---|---|---|
| Active groups | reaction | of the “old tooth replacement”/ |
| of the Bonding agent → | with → | the incisal material |
| —OH | ″ | —CO₂H |
| —NCO | ″ | —OH; —CO₂H; —NHR; —SH |
| —NHR | ″ | C═C double bond (activated), |
| | | —CO₂H |
| Activated —CO₂X | ″ | —OH; —NHR; —SH |
| —SH | ″ | C═C double bond |
| | | (activated or not activated) |
| C═C double bond | ″ | C═C double bond |
| (activated or not activated) | | (activated or not activated), |
| | | —SH |
| C═C (activated) | ″ | —NHR |
| Epoxide; cyclical carbonate | ″ | —OH; —CO₂H; —NHR; —SH |

Groups having these types of double bonds are referred to as “activated C═C double bonds”, in the vicinity of which there is an electron-withdrawing group, such that an attack by an —NHR group (a nucleophilic attack) is possible. Examples for these radicals are acrylates and methacrylates. Instead of the term “activated C═C double bonds”, in some areas below, the term “active C═C double bonds” is also used.

In a first embodiment, in which the repair or incisal material is comprised of a material having free hydroxy groups, said functional groups of these compounds may selected from among carboxyl (carboxylic acid or activated carboxylic acid groups, such as anhydride groups), epoxy or isocyanate groups. Without wanting to be attached to a theory, the inventors assume that this effect is based on the bond to remaining OH groups on the otherwise no longer reactive surface of the “old” dental replacement. These OH groups may have various origins—either the “old” dental replacement is comprised of a material having organically-bonded free hydroxy groups (e.g. in silicic acid polycondensates produced from silanes of a formula (B) with R²=OH) or silicon-bonded hydroxy groups (e.g. with incomplete condensation of the silanes following complete hydrolysis), or said OH groups can be subjected to mechanical-chemical measures, such as an etching process with an aqueous, acid or alkaline medium, through which, for example, Si—O—Si bridges were split through the formation of Si—OH groups or ester groups were split through the formation of free, organically-bonded hydroxy groups.

Examples for bonding agent molecules containing isocyanate, which can be used in the first embodiment, are dicyclohexylmethane diisocyanate, Hexamethylene-1,6-diisocyanate, Hexamethylene-1,8-diisocyanate, Diphenylmethane-4,4-diisocyanate, Diphenylmethane-2,4-diisocyanate, 2,4-toluene diisocyanate, 2,6-toluene diisocyanate, Triphenylmethane-4,4′,4″-triisocyanate, 3-Isocyanatomethyl-3,5,5-trimethylcyclohexyl diisocyanate, and tris(p-isocyanatophenyl)thiophosphate.

In a second embodiment, said repair or incisal material is comprised of a material having free, potentially activated carboxylic acid groups, and a compound having at least two isocyanate groups is used as a bonding agent. This then reacts on the one hand with said OH groups of the “old” material and on the other hand with the carboxylic acid groups of the repair or incisal material.

In a third embodiment, in which the repair or incisal material is comprised of a material having groups containing C═C double bonds, said functional groups of the specified difunctional compounds can be selected from among thiol and amino groups, and in this case, it is necessary that the material of the “old” dental replacement has C═C double bonds as well, as is the case, e.g. with all materials (A) to (G) depicted above. If said functional groups are amino groups, an additional condition must be complied with, namely the specified C═C double bonds must be activated in both materials, that of the “old” dental replacement and that of the repair or incisal material, e.g. through the presence of an electron-withdrawing group. Examples for these radicals are acrylates and methacrylates.

Examples for bonding agent molecules containing amino groups, which can be used in this third embodiment, are diaminoacetone, diaminoacridine, diaminoadamantane, diaminoanthraquinone, benzidine, diaminobenzoic acid, phenylenediamine, diaminobenzophenone, diaminobutane, diaminocyclohexane, diaminodecane, diaminodicyclohexylmethane, diamino-methoxybiphenyl, diamino-dimethylhexane, diaminodiphenylmethane, diaminododecane, diaminoheptane, diaminomesitylene, diaminomethylpentane, diaminomethylpropane, naphtyhlenediamine, diaminoneopentane, diaminooctane, diaminopentane, diaminophenanthrene, diaminopropane, diaminopropanol, diaminopurine, and diaminopyrimidine.

Examples for bonding agent molecules containing thio groups, which can be used in this third embodiment, are Trimethylolpropane tri(3-mercaptopropionate) (TMPMP); trimethylolpropane trimercaptoacetate) (TMPMA); Pentaerythritol tetra(3-mercaptopropionate) (PETMP); pentaerythritol tetramercaptoacetate) (PETMA); glycol dimercaptoacetate; Glycol di(3-mercaptopropionate); ethoxylated Trimethylolpropane tri(3-mercaptopropionate); Biphenyl-4-4′-dithiol; p-Terphenyl-4,4″-dithiol; 4,4′-Thiobisbezenthiol; 4,4′-Dimercaptostilbene; Benzene-1,3-dithiol; Benzene-1,2-dithiol; Benzene-1,4-dithiol; 1,2-Benzenedimethanethiol; 1,3-Benzenedimethanethiol; 1,4-Benzenedimethanethiol; 2,2″-(Ethylenedioxy)diethanethiol; 1,6-Hexanedithiol; 1,8-Octanedithiol, and 1,9-Nonanedithiol

It should be particularly noted, that moisture in the mouth does not have a detrimental impact if thiols or isocyanates are used as a bonding agent. With the adhesive systems normally used in the state of the art for attaching composite fillings on a tooth, the entire area must be dried as the bonding effect is negatively impacted through moisture. This step is normally omitted with the bonding agents pursuant to the invention with the specified groups.

Schematically, the bonding agent can be explained based on the following diagram:

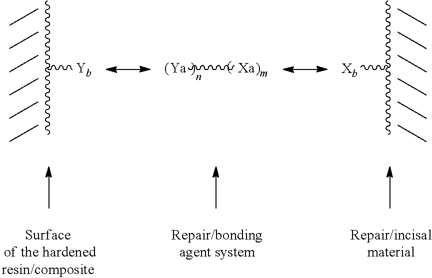

The reactive groups, which are still located on the surface of the “old” dental replacement, are designated by Yb. Ya designates the functional group of said difunctional compound, which should be reacted with it. Xa is the second functional group of the difunctional compound, which is selected with respect to the groups present on the repair composite (in this case, designated by “repair/incisal material”). Its reactive groups are labeled with Xb. n and m respectively independently stand for at least 1, however, they can also be 2, 3, 4 or even greater.

With the present invention, it may be beneficial if said difunctional compound has two or at least two identical groups, i.e. Ya and Xa are identical. In the event that the “old” dental replacement essentially or only has free hydroxy groups on its surface (as is often the case for the surface of the silicic acid (hetero) polycondensate, i.e. Yb=OH) due to the hardening and/or surface processing, this involves dicarboxylic acids, bisanhydrides, bisepoxides or diisocyanates—potentially substituted with additional, even possibly reactive groups—i.e. Ya is —NCO, epoxy or —COA′ with A′=an element, which forms an anhydride radical with said —CO group. In these cases, a particularly good bond is achieved if a resin system is used for the repair material, which was made from silanes containing hydroxy groups, for example, a resin system made from or through the use of silanes of a formula (A), which additionally have free hydroxy groups, or silanes of a formula (B), in which R² is equal to OH (Xb=OH). Naturally, it is beneficial if the “old” dental replacement was likewise made from this or a comparable resin system—in the best case scenario, the number of said OH groups remaining on its surface is therefore greater; in the worst case, this will have no impact. If the “old” dental replacement contains remaining C═C double bonds, these can be used for bonding as well, as previously mentioned, for example, with the help of a difunctional compound, which contains thiol groups or (if said C═C double bonds are present in a configuration, in which they are activated, see above) amino groups. Due to the fact that there are many suitable repair/incisal materials likewise having C═C double bonds (which are indeed at least partially used through hardening after being applied), dithiol compounds (or potentially diamines) are beneficial as difunctional compounds for such systems. With regard to the diagram above, in these cases Xb and Yb stand for respectively one group, which has a (potentially activated) C═C double bond, while Xa and Ya represent SH or NH₂ or NHR with R=e.g. alkyl, aryl, alkylaryl having preferably 1 to 6 carbon atoms for non-arylized groups and preferably 6-16 carbon atoms for arylized groups.

Rather, it will occur that the dentist or dental technician chooses another repair material, which e.g. has a different hardness than that of the “old” dental replacement, or that an incisal material differing from the base body should be applied. A difunctional compound must then be chosen, the group or groups Xa of which can react with reactive groups of the repair material. In one embodiment, the invention suggests that these groups are identical to groups Ya, e.g. in the event of a repair material having free carboxylic groups—then said groups Xa can be, for example, epoxy groups or isocyanate groups just as groups Ya. However, in another embodiment, said groups Ya and Xa can also be different. Then it is possible to select these independent from each other freely under the aspect that at least one thereof can react with the “old” dental replacement and at least another can react with reactive groups of said repair material. With reference to the diagram above, which shows the reactions of certain functional groups with active groups on the surface of the “old” dental replacement/said incisal material, it becomes clear which possibilities there are of selecting the functional groups of the bonding agent according to the presence of the active groups on both sides of the materials. For example, if the “old” dental replacement has groups with C═C double bonds and said incisal material is comprised of a silicic acid (hetero) polycondensate, which has CO₂H groups in addition to groups with those non-cross-linked C═C double bonds, a substance, which has one or two SH and one or two OH groups, can be used as a bonding agent. Examples for this are thioglycerol (3-Mercaptopropane-1,2-diol), 6-Mercapto-1-hexanol, 11-Mercapto-1-undecanol, and 1-Mercaptoundec-11-yl)-tetraethylene glycol. If the same system is used, wherein however the substance of said incisal material has free hydroxy groups instead of carboxylic acid groups, a substance can be used as a bonding agent, which has one or two SH groups and one or two COOH groups. Examples are 11-mercaptoundecanol acid, 3-mercaptopropionol acid, 3- or 4-mercaptophenyl acetic acid, 16-mercaptohexadecanoic acid, 8-mercaptooctane acid, 15-Mercaptopentadecanoic acid, and 4-mercaptophenyl acetic acid. Other examples for bonding agents with different groups Ya and Xa are: methacrylic acid isocyanatoethyl ester and glycidyloxypropyl methacrylate (reacting with OH—, CO₂H—, NHR—, and SH groups as well as double bonds), ethanolamine, 2-methyl ethanolamine, and diethanolamine (reacting with activated C═C double bonds as well as CO₂H).

In one particular embodiment, one of the (at least) two functionalities of the bonding agent may be a mono, di or trialkoxysilane group instead of a group as listed above. It bonds to Si—OH groups in the “old” dental replacement or said incisal material. The at least one other functionality may then be, for example, an SH group. Examples are 3-mercaptopropyltrimethoxysilane, 3-mercaptopropyltriethoxysilane, and 3-mercaptopropylmethyldimethoxysilane. Silanes of this type can be used even in a hydrolytically partially condensed form. This then involves a hydrolytically partially condensed, organically-modified silicic acid poly (partial) condensate, for which a part of the alkoxy groups of said silane has passed into Si—O—Si structures through condensation, while another part of said alkoxy groups was hydrolyzed or remained unchanged. Therefore, there are both Si—OR (with OR=alkoxy) as well as Si—OH groups in these silicic acid poly (partial) condensates. These partial condensates are produced through careful hydrolysis with an insufficient amount of hydrolysis reagent. They are comparable to said monomeric compounds insofar as they bear at least one additional group, such as the mercapto group, per silyl group and their Si—OH groups act like said alkoxy groups, though they are somewhat more reactive than these.

The backbone of said difunctional compound, which bonds both functional groups, can be selected randomly. For example, it can be a hydrocarbon potentially interrupted by linkage groups, such as COO, CONH, NHCO or the like or oxygen or sulfur atoms or NH groups. It can be selected from among preferably straight-chained (though branched or cyclical as well), alkylene groups, respective alkenylene groups, arylene groups, aralkylene groups, alkylarylene groups or alkyl-arylalkylene groups. The number of carbon atoms contained therein is not critical and can, for example, be between 2 and 6 for non-aromatic and non-cyclical compounds and 5 to 20 for cyclical compounds or those containing aryl groups. In another embodiment, said difunctional compound is a silane having two radicals bonded to said silicon via carbon respectively bearing one of the necessary functional groups, or having such a radical, which bears both of the necessary functional groups, or a disilane having such groups or a silane resin (a condensate) made from such silanes.

Of course, it is beneficial if said difunctional compound is a liquid, e.g. hexamethylene diisocyanate. However, it can also be applied as a solution to the surface of the “old” dental replacement, such that solid or paste-like compounds can also be used if they are dissoluble in a suitable solvent. Therefore, in the case of isocyanates, primarily esters, such as acetic aster, or ethers, such as tetrahydrofuran, are worth consideration. If said difunctional compound is not contacted by water, water-alcohol mixtures as well as alcohols, these solvents are worth consideration as well.

If filled silicic acid (hetero) polycondensates are used for the “old” dental replacement and/or the repair or incisal material, the filler can also be used for an improved bond of both materials in special cases. This is achieved if the particles used for the filling also have free groups, which can be used for binding the bonding agent. Examples are fillers bearing SiOH groups on their surface, such as Aerosil or silanized filler particles. If silanized particles bear ester groups on their surface, they can be split through the formation of OH or OH and COOH groups, just as Si—O—Si, as is described above in connection with the effect of etching of the surface of the “old” dental replacement.

In one particular embodiment of the invention, which can be combined with all of the aforementioned embodiments, a color adjustment is made during the repair of the dental replacement or crown base body.

It is common that crowns or certain areas thereof are painted with color pastes/stains in order to achieve highest possible esthetics and thus to look real. As a final step, these stains are applied, e.g. to crowns made from an organic plastic, and hardened by means of, e.g. blue light emitters. A great disadvantage in this case is that these color pastes are not very resistant to abrasions. By rubbing together when chewing, the applied layers of colors are removed very quickly. Thus, the color effect wears off and the crown loses its esthetics.

To help this, the invention suggests not applying or not only applying the stains to the outer layer. Due to the multilayered structure of the composite crowns pursuant to the invention, it is namely possible to apply stains between the outer (enamel layer/incisal material) and the base body.

In a first design of this embodiment, an additional intermediate layer is applied. In this process, a bonding agent is first applied to the potentially roughened base body. A color paste comprising a matrix system with a photo initiator, which corresponds to that of the enamel layer (i.e. having the same organically-modified silicic acid (hetero) polycondensate as the enamel layer or having such a silicic acid (hetero) polycondensate that it has the same reactive groups as said enamel layer), as well as one or more color pigments (e.g. titanium dioxide, iron oxide in a quantity of preferably 0.001-1.0 per mill) is applied and hardened with blue light. The polymerized stain acts as a dispersion layer in the process making a second application of a bonding agent unnecessary. Subsequently, the enamel layer can now be applied and hardened.

An additional design provides that one or more color pigments are mixed directly into the bonding agent (e.g. titanium dioxide, iron oxide—quantity preferably in an amount of 0.001-1.0 per mill). Said bonding agent is applied to the potentially roughened base body. Subsequently, the enamel layer can be applied and hardened with blue light.

In a third design, the color pigments are added to “classic” purely organic monomers or prepolymers, as they are used for the production of organic dental systems. Examples for these types of monomers were previously specified above, including Bis-GMA having OH and C═C groups, UDMA and TEGDMA having only C═C groups. However, e.g. mixtures of these monomers are possible as well. The quantity of color pigment is in the same range as specified for the bonding agent. Said monomers are applied to the existing dental replacement after roughening and/or prior to applying the bonding agent.

Potentially two or, if desired, even all three of the aforementioned designs can be applied in combination.

In both cases, the stains/coloring pastes are protected through the outer enamel layer. Thus, they are prevented from being quickly worn down when chewing. The esthetic remains intact.

The invention is not limited to the field of dentistry—it can potentially be used for applying a silicic acid (hetero) polycondensate or a composite made from such a filler-filled condensate on the surface of a previously hardened, unfilled or filler-filled silicic acid (hetero) polycondensate (or even purely organic materials; see the introductory description of the material) of any form and in any technical contexts. Examples are (micro)optic as well as (micro)electronic application.

The invention will be explained in further detail based on the following design examples:

### I. RESIN SYSTEMS FOR THE “OLD” TOOTH MATERIAL

Synthesis of Resin System a (from DE 44 16 857)

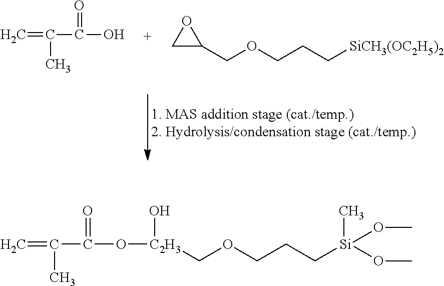

For receiving 125.0 g (0.503 mol) of 3-Glycidyloxypropyltrimethoxysilane, triphenylphosphine (as a cat.), BHT (as a stabilizer), and then 47.35 g (0.550 mol) of methacrylic acid are added drop-wise in a dry atmosphere and stirred at 80° C. (approx. 24 hrs.). The reaction can be followed by the decrease in the concentration of carboxylic acid via acid titration and the epoxy conversion can be followed via Raman spectroscopy/epoxy titration. The band of epoxy silane characteristic for the epoxy group appears in the Raman spectrum at 1256 cm⁻¹. The epoxy or carboxylic acid conversion is at 99% or 89% (→because 1:1.1 is a carboxylic acid surplus). After adding acetic ester (1000 ml/mol of silane) and H₂O for hydrolysis with HCl (as a cat.), it is stirred at 30° C. The progress of the hydrolysis is respectively followed via water titration. Processing occurs approximately after multiple days of stirring through repeated extraction with aqueous NaOH and with water and filtration via hydrophobized filters. A rotary evaporator is used first and then an oil pump vacuum is used for suctioning. This results in a liquid resin without the use of reactive thinners (monomers) having a very low viscosity of approx. 3-6 Pa·s at 25° C. (heavily dependent upon exact hydrolysis and processing conditions) and 0.00 mmol of CO₂H/g (no free carboxyl groups).

It should be noted that all aforementioned bonding agents can be used with this material because it has both (activated) C═C double bonds as well as (organically-bonded) hydroxyl groups.

If condensation of said silanes does not occur entirely, additional SiOH groups will remain intact in the material and therefore on its surface as well. This applies for all hydrolysable and condensable silane materials.

### Synthesis of Resin System D (DE 10 2011 053 865.8)

### Basic Reaction Principle:

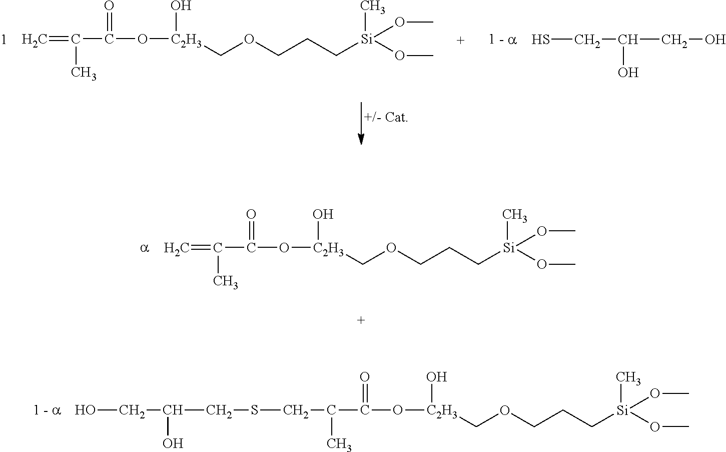

### Example of the 1st Reaction:

For receiving 80.4 g (0.18 mol) of Resin System A and potentially 0.17 g of triethylamine, 8.96 g (0.276 mol) of 3-Mercaptopropane-1,2-diol are added drop-wise while stirring. The reaction can be followed via NMR and by the decrease of an HS band via IR spectrum. The band characteristic for said HS group appears in the Raman spectrum at 2566 cm⁻¹. The result is a liquid resin having a viscosity of approx. 16-18 Pa·s at 25° C. (depending on the exact synthesis and processing conditions of the preliminary stages). Additional processing is normally not necessary.

### Synthesis of Resin System E (DE 44 16 857)

For receiving 129.2 g (0.52 mol) of 3-Glycidyloxypropyltrimethoxysilane, triphenylphosphine (as a cat.), BHT (as a stabilizer), and then 47.35 g (0.550 mol) of methacrylic acid are added drop-wise in a dry atmosphere and stirred at approx. 80° C. (approx. 24 h). The reaction can be followed by the decrease in the concentration of carboxylic acid via acid titration and the epoxy conversion can be followed via Raman spectroscopy/epoxy titration. The band of epoxy silane characteristic for the epoxy group appears in the Raman spectrum at 1256 cm⁻¹. After adding acetic ester (1000 ml/mol of silane) and H₂O for hydrolysis with HCl (as a cat.), it is stirred at 30° C. and 22.2 g (0.10 mol) of methacryloxy-methyltrimethoxysilane are added slowly drop-wise. The progress of the hydrolysis is respectively followed via water titration. Processing occurs after two days of stirring through repeated extraction with aqueous NaOH and with water and filtration via hydrophobized filters. A rotary evaporator is used first and then an oil pump vacuum is used for suctioning. This resulted in a liquid resin without the use of reactive thinners (monomers) having a viscosity of approx. 45.6 Pa·s at 25° C. (heavily depending upon the exact synthesis and processing conditions).

Resin System E differs from Resin System A in that the output materials subjected to hydrolytic condensation additionally have methacryloxy-methyltrimethoxysilane, which leads to a stronger inorganic cross-linking of the resulting system.

### Ia Composites for Crowns with a High Breaking Strength (Preferably with a Breaking Strength, which is Greater than Those of PMMA-Based Materials (Approx. 93 MPa)

### Example Ia-1

50% of Resin System A by weight+2% DBPO
50% Silmikron 810-10/1 filler by weight (comprised of SiO₂ by nearly 99% by weight), primary particle size: 0.5 μm, non-silanized (company: Quarzwerke)
Incorporation of the filler: 3 passes in a three-roll mill
Thermal hardening for 4 hours at 100° C.; 1 day of dry storage at 40° C.
Breaking strength: 100±5 MPa
Modulus of elasticity: 4.5±0.11 GPa

### Example Ia-2

50% of Resin System A by weight+2% DBPO
50% Silbond 960-943 MST filler by weight (comprised of SiO₂ by nearly 99% by weight), primary particle size: 1.2 μm, silanized (company: Quarzwerke)
Incorporation of the filler: 3 passes in a three-roll mill
Thermal hardening for 4 hours at 100° C., 1 day of dry storage at 40° C.
Breaking strength: 101±11 MPa
Modulus of elasticity: 5.1±0.08 GPa

### Example Ia-3

50% of Resin System A by weight+2% DBPO
50% Silbond FW 600 MST filler by weight (comprised of SiO₂ by nearly 99% by weight), primary particle size: 4 μm, silanized (company: Quarzwerke)
Incorporation of the filler: 3 passes in a three-roll mill
Thermal hardening for 4 hours at 100° C., 1 day of dry storage at 40° C.
Breaking strength: 113±8 MPa
Modulus of elasticity: 5.40±0.05 GPa

### Example Ia-4

40% of Resin System A by weight+2% DBPO
60% Silbond FW 600 MST by weight (comprised of SiO₂ by nearly 99% by weight), primary particle size: 4 μm, silanized (company: Quarzwerke)
Incorporation of the filler: 2× three-roll mill
Thermal hardening for 4 hours at 100° C., 1 day of dry storage at 40° C.
Breaking strength: 126±10 MPa
Modulus of elasticity: 7.3±0.11 GPa

### Example Ia-5

40% of Resin System A by weight+2% DBPO
60% of a filler mixture by weight (company: Quarzwerke)
Silmikron 810-10/1 (comprised of SiO₂ by nearly 99% by weight), primary particle size: 0.5 μm, non-silanized (2 passes in a three-roll mill)
Silbond FW 600 MST (comprised of SiO₂ by nearly 99% by weight), primary particle size: 4 μm, silanized (2×15 min. in a planetary mixer)
   Thermal hardening for 4 hours at 100° C., 1 day of dry storage at 40° C.
   Breaking strength: 131±14 MPa
   Modulus of elasticity: 7.0±0.14 GPa

### Example Ia-6

40% of Resin System A by weight+2% DBPO
60% Silmikron 810-10/1 filler by weight (comprised of SiO₂ by nearly 99% by weight), primary particle size: 0.5 μm non-silanized (company: Quarzwerke)
Incorporation of the filler: 3 passes in a three-roll mill
Thermal hardening for 4 hours at 100° C., 1 day of dry storage at 40° C.
Breaking strength: 148±8 MPa
Modulus of elasticity: 6.4±0.17 GPa

### Ib Additional Composites for Permanent Crowns/Base Material (Essential Aspects in this Case are: A High Breaking Strength of Preferably More than 113±10 MPa and a High Modulus of Elasticity, a High Level of Fracture Toughness, a High Degree of Hardness, and Minimal Abrasion)

### Example Ib-1

15% of Resin System A by weight+1.5% DBPO
85% of a filler mixture by weight, silanized (55% SiO₂ by weight, 25% BaO by weight, 10% B₂O₃ by weight, 10% Al₂O₃ by weight) (Schott glass GM 27884), comprised of:
18% nanofine, primary particle size: 0.18 μm (1 pass in a three-roll mill)
14% ultrafine, primary particle size: 0.40 μm (1 pass in a three-roll mill)
68% K6, primary particle size: 3.0 μm (2×15 min. in a planetary mixer, 20 RPM)
   Thermal hardening for 4 hours at 100° C., 1 day of dry storage at 40° C.
   Breaking strength: 167±15 MPa
   Modulus of elasticity: 13.8±0.66 GPa
   Vickers hardness: 100 HV 0.5; 30 s

### Example Ib-2

100% of Resin System D by weight+1.2% of camphor quinone+1.8% DABE
Photo-initiated hardening for 100 seconds on both sides, 1.5 days of dry storage at 40° C.
Breaking strength: 130±4 MPa
Modulus of elasticity: 2.7±0.09 GPa

### Example Ib-3

100% of Resin System D by weight+1% of Lucirin TPO
Photo-initiated hardening for 100 seconds on both sides, 1.5 days of dry storage at 40° C.
Breaking strength: 132±4 MPa
Modulus of elasticity: 2.7±0.11 GPa

### Example Ic Single-Color Crowns

### Example Ic-1

15% of Resin System A by weight+1.5% DBPO
85% of a filler mixture by weight, silanized (55% SiO₂ by weight, 25% BaO by weight, 10% B₂O₃ by weight, 10% Al₂O₃ by weight) (Schott glass GM 27884), comprised of:
18% nanofine, primary particle size: 0.18 μm (1 pass in a three-roll mill)
14% ultrafine, primary particle size: 0.40 μm (1 pass in a three-roll mill)
68% K6, primary particle size: 3.0 μm (2×15 min. in a planetary mixer, 20 RPM)
Thermal hardening for 3 hours at 100° C.

### Example Ic-2

30% of Resin System A by weight+2% DBPO
70% filler by weight, non-silanized 0.7 μm (65% SiO₂ by weight, 15% B₂O₃ by weight, <5% Al₂O₃ by weight, 5% K₂O by weight, 10% Cs₂O₃ by weight, 5% La₂O₃ by weight, <5% ZrO₂ by weight) (Schott glass GO 18-307)
Incorporation of the filler: Combination of the three-roll mill and planetary mixer
Thermal hardening for 4 hours at 100° C.

### Id Composites for Fillings

These composites can be manufactured from the same materials as the aforementioned crown composites.

### Ie Composites for the Repair System/Said Incisal or Repair Material: (in this Case, the Significant Aspects are a High Level of Esthetics, a High Level of Translucency, a High Degree of Hardness, Minimal Abrasion, and the Ability to be Polished Well)

### Example Ie-1

71.4% of Resin System A by weight+2% DBPO
28.6% Silmikron 810-10/1 by weight (comprised of SiO₂ by nearly 99% by weight), primary particle size: 0.5 μm, non-silanized (company: Quarzwerke)
Incorporation of the filler: 1 pass in a three-roll mill
Thermal hardening for 4 hours at 100° C., 1 day of dry storage at 40° C.

| Breaking | Modulus of | Translucence | C═C |
|---|---|---|---|
| strength [MPa] | elasticity [GPa] | [%] | reaction [%] |
| 98 ± 8 | 3.1 ± 0.09 | 42 | 93 |

### Example Ie-2

71.4% of Resin System A by weight+2% DBPO
28.6% Trisopor 4000 filler by weight, non-silanized, porous glass, (267 nm pore size), amorphous, at least 90% SiO₂ (company: VitraBio)
Incorporation of the filler: 3 passes in a three-roll mill
Thermal hardening for 4 hours at 100° C., 1 day dry storage at 40° C.

| Breaking | Modulus of | Translucence | C═C |
|---|---|---|---|
| strength [MPa] | elasticity [GPa] | [%] | reaction [%] |
| 111 ± 7 | 3.5 ± 0.07 | 41 | 98 |

### Example Ie-3

71.4% of Resin System A by weight+2% DBPO
28.6% Ultrafine filler by weight, primary particle size: 0.40 μm, silanized (55% SiO₂ by weight, 25% BaO by weight, 10% B₂O₃ by weight, 10% Al₂O₃ by weight) (Schott glass GM 27884) Incorporation of the filler: 1 pass in a three-roll mill
Thermal hardening for 4 hours at 100° C., 1 day of dry storage at 40° C.

| Breaking | Modulus of | Translucence | C═C |
|---|---|---|---|
| strength [MPa] | elasticity [GPa] | [%] | reaction [%] |
| 100 ± 5 | 2.9 ± 0.05 | 53 | 94 |

### Example Ie-4

71.4% of Resin System A by weight+2% DBPO
28.6% Trisopor 400 filler by weight, non-silanized, porous glass (40 nm pore size), amorphous, mind. 90% SiO₂) (company: VitraBio)
Incorporation of the filler: 2 passes in a three-roll mill
Thermal hardening for 4 hours at 100° C., 1 day of dry storage at 40° C.

| Breaking | Modulus of | Translucence | C═C |
|---|---|---|---|
| strength [MPa] | elasticity [GPa] | [%] | reaction [%] |
| 104 ± 7 | 3.6 ± 0.06 | 76 | 95 |

### Example Ie-5

50% of Resin System A by weight+2% DBPO
50% nanofine filler by weight, primary particle size: 0.18 μm, silanized (55% SiO₂ by weight, 25% BaO by weight, 10% B₂O₃ by weight, 10% Al₂O₃ by weight) (Schott glass GM 27884)
Incorporation of the filler: 1 pass in a three-roll mill, subsequently: vacuum process
Thermal hardening for 4 hours at 100° C., 1 day of dry storage at 40° C.

| Breaking | Modulus of | Translucence | C═C |
|---|---|---|---|
| strength [MPa] | elasticity [GPa] | [%] | reaction [%] |
| 127 ± 7 | 4.4 ± 0.04 | N.A. | N.A. |

### Example Ie-6

75% of Resin System A by weight+2% DBPO
25% spray-dried nanoparticle filler by weight, primary particle size: 70 nm, non-silanized (produced pursuant to DE 10 2005 061965). Incorporation of the filler: 1 pass in a three-roll mill Thermal hardening for 4 hours at 100° C., 1 day of dry storage at 40° C.

| Breaking | Modulus of | Translucence | C═C |
|---|---|---|---|
| strength [MPa] | elasticity [GPa] | [%] | reaction [%] |
| 80 ± 3 | 2.4 ± 0.05 | N.A. | N.A. |

### Example Ie-7

30% of Resin System E by weight+2% DBPO
70% of a filler mixture by weight, silanized (65% SiO₂ by weight, 15% B₂O₃ by weight, <5% Al₂O₃ by weight, 5% K₂O by weight, 10% Cs₂O₃ by weight, 5% La₂O₃ by weight, <5% ZrO₂ by weight) (Schott glass G018-307), comprised of:
25% ultrafine, primary particle size: 0.7 μm (3 passes in a three-roll mill)
75% K5, primary particle size: 5.0 μm (2×15 min. in a planetary mixer, 40 RPM, 1×15 min. with 0.8 bar of negative pressure (degassing)
   Thermal hardening for 4 hours at 100° C., 1 day of dry storage at 40° C.
   Breaking strength: 129±9 MPa
   Modulus of elasticity: 10.2±0.14 GPa

### Example Ie-8

30% of Resin System E by weight+1% of Lucirin TPO
70% of a filler mixture by weight, silanized (65% SiO₂ by weight, 15% B₂O₃ by weight, <5% Al₂O₃ by weight, 5% K₂O by weight, 10% Cs₂O₃ by weight, 5% La₂O₃ by weight, <5% ZrO₂ by weight) (Schott glass G018-307), comprised of:
25% ultrafine, primary particle size: 0.7 μm (3 passes in a three-roll mill)
75% K5, primary particle size: 5.0 μm (2×15 min. in a planetary mixer, 40 RPM, 1×15 min. with 0.8 bar of negative pressure (degassing)
   Photo-initiated hardening for 100 seconds on both sides, 1.5 days of dry storage at 40° C.
   Breaking strength: 123±9 MPa
   Modulus of elasticity: 8.9±0.44 GPa

### II. COMPOSITIONS OF THE COMPOSITE FOR SHEAR STRENGTH TESTS

For the following examples, crowns or a filling composite were used as a base material and the repair composite was used as a subsequently applied material. The compositions are listed in Example II-1 to II-3. Naturally, the listed composite compositions serve merely as examples and should not be limited to the invention.

### Example II-1

The crown composite used for the measurements is comprised of Resin System A, 2% (dibenzoyl peroxide) DBPO and 70% K6 filler by weight, primary particle size 3 μm (GM 27884, manufactured by Schott, composition: 55% SiO₂ by weight, 25% BaO by weight, 10% B₂O₃ by weight, 10% Al₂O₃ by weight). The filler was incorporated in a planetary mixer twice for 15 minutes at 30 RPM and subsequently once for degassing for 15 minutes with 0.8 bar of negative pressure. The crown composite was polymerized for 4 hours at 100° C. in order to determine the shear strength.

### Example II-2

The filling composite used for the measurements is comprised of Resin System A, 0.6% camphor quinone (CC) and 0.9% dimethylamino benzoic acid ethyl ester (DABE), and 70% K6 filler by weight, primary particle size 3 μm (GM 27884, manufactured by Schott, composition: 55% SiO₂ by weight, 25% BaO by weight, 10% B₂O₃ by weight, 10% Al₂O₃ by weight). The filler was incorporated in a planetary mixer twice for 15 minutes at 30 RPM and subsequently once for degassing for 15 minutes with 0.8 bar of negative pressure. The filling composite was polymerized on both sides for respectively 120 seconds using blue light in order to determine the shear strength.

### Example II-3

The repair composite used for the measurements is comprised of Resin System A, 0.6% CC and 0.9% DABE and 70% of a filler mixture by weight comprised of: 15% nanofine 180 (primary particle size 0.18 μm), 14% ultrafine 400 (primary particle size 0.40 μm), 71% K6 (primary particle size 3.0 μm), (GM 27884, manufactured by Schott, composition: 55% SiO₂ by weight, 25% BaO by weight, 10% B₂O₃ by weight, 10% Al₂O₃ by weight). Said fillers, nanofine 180 and ultrafine 400, were incorporated respectively with 2 passes in a three-roll mill at 130 RPM. The filler was incorporated in a planetary mixer twice for 15 minutes at 30 RPM and subsequently once for degassing for 15 minutes with 0.8 bar of negative pressure. The repair composite was hardened as specified in the design examples.

### III. COMPARATIVE MEASUREMENTS OF PURELY MECHANICALLY PRETREATED COMPOSITES (SURFACE ENLARGEMENT THROUGH SANDBLASTING)

Shear strength samples were produced for the comparative measurements, consisting of a cylindrical base material (crown composite) and a smaller cylinder made of repair composite centrically attached to one of its surfaces. By cutting the smaller cylinder, we determined in what area the shear strength of the material lies. The crown composite is preferably thermally hardened and thus corresponds to said material, which is normally inserted for crown moldings—see above. Said repair composite was normally hardened photo-induced due to the possibility of conducting the repair directly in the mouth of the patient. Crown and filling composites form the base material, onto which the repair composite is applied.

### Example III-1

Surface of the crown composite was blasted with corundum (50 μm grain size) with 2.8 bar of pressure at a vertical distance of 1 cm from the surface, with the help of a silicon ring, the cylinder from the repair composite was polymerized to the crown composite in two steps for respectively 60 seconds with blue light; 1 day of storage at 40° C., dry and dark; shear strength: 11±2 MPa (purely adhesive failure between the composites)

### Example III-2

Surface of the crown composite was blasted with corundum (110 μm grain size) with 2.8 bar of pressure at a vertical distance of 1 cm from the surface, with the help of a silicon ring, the cylinder from the repair composite was polymerized to the crown composite in two steps for respectively 60 seconds with blue light; 1 day of storage at 40° C., dry and dark; shear strength: 13±1 MPa (purely adhesive failure between the composites)

### Example III-3

Surface of the crown composite was blasted with corundum (150 μm grain size) with 2.8 bar of pressure at a vertical distance of 1 cm from the surface, with the help of a silicon ring, the cylinder from the repair composite was polymerized to the crown composite in two steps for respectively 60 seconds with blue light; 1 day of storage at 40° C., dry and dark; shear strength: 12±1 MPa (adhesive failure with one third of adhesive failures, i.e. minimal portions of the bonded area partially demonstrate cohesive breaking out of the crown composite)

### Example III-4

Surface of the crown composite was blasted with corundum (250 μm grain size) with 2.8 bar of pressure at a vertical distance of 1 cm from the surface, with the help of a silicon ring, the cylinder made of repair composite was polymerized to the crown composite in two steps for respectively 60 seconds with blue light; 1 day of storage at 40° C., dry and dark; shear strength: 15±1 MPa (adhesive failure with two thirds of adhesive/cohesive failures, i.e. minimal portions of the bonded area partially demonstrate cohesive breaking out of the crown composite)

These comparative measurements have demonstrated that the bond to the crown composite increases with a greater grain size of the blasting sand. Without this pretreatment, a sufficient bond could not be achieved because the cylinders to be cut already came loose from the surface when demolding from the silicon ring. The blasting sand having a grain size of 150 and 250 μm even caused that small areas of the shearing area cohesively, i.e. that the basic material and not the bond, fail. Through the use of bonding agents, a further increase of the bond should be brought about.

In the following examples, no shear strengths are provided for purely cohesive failures in the composite as this does not focus on the actual bonding value, but rather the strength of the composite. Once there is a cohesive fracture, this shows that the bond is stronger than the composite itself.

### IV SYNTHESIS OF VARIOUS BONDING AGENTS WITH AT LEAST TWO DIFFERENT FUNCTIONAL GROUPS

### Example IV-1

### Production of a Silicic Acid Poly (Partial) Condensate with Methacrylate Groups

1 g 3-Trimethoxysilyl propyl methacrylate is mixed with 19 g of acetone. While stirring, 0.109 g of 1N HCl is added and continually stirred for 2 hours.

### Example IV-2

### Producing a Silicic Acid Poly (Partial) Condensate with Methacrylate and Carbon-Bonded OH Groups

For receiving 25.3 g (0.102 mol) of 3-Glycidyloxypropyl(methyl) diethoxysilane, 0.26 g of triphenylphosphine (as a cat.), 0.02 g of BHT (as a stabilizer), and then 8.61 g (0.100 mol) of methacrylic acid are added drop-wise in a dry atmosphere and stirred at 85° C. (approx. 24 hours). The reaction can be followed by the decrease in the concentration of carboxylic acid via acid titration and the epoxy conversion can be followed via Raman spectroscopy/epoxy titration. The band of epoxy silane characteristic for the epoxy group appears in the Raman spectrum at 1256 cm⁻¹. 1 g of the product is mixed with 19 g of acetone. 0.108 g of 1N HCl is added while stirring and continually stirred for 0.5 hours.

### Example IV-3

### Producing a Silicic Acid Poly (Partial) Condensate with Methacrylate and Carbon-Bonded OH Groups

For receiving 28.7 g (0.122 mol) of 3-Glycidyloxypropyl trimethoxysilane, 0.31 g of triphenylphosphine (as a cat.), 0.024 g of BHT (as a stabilizer), and then 10.33 g (0.120 mol) of methacrylic acid are added drop-wise in a dry atmosphere and stirred at 85° C. (approx. 24 hours). The reaction can be followed by the decrease in the concentration of carboxylic acid via acid titration as well as the epoxy conversion can be followed via Raman spectroscopy/epoxy titration. The band of epoxy silane characteristic for the epoxy group appears in the Raman spectrum at 1256 cm⁻¹.

1 g of the product is mixed with 19 g of acetone. 0.168 g of 1N HCl is added while stirring and continually stirred for 0.5 hours.

### V. EXAMPLES FOR THE MEASUREMENT OF THE BOND OF THE REPAIR COMPOSITE TO THE CROWN COMPOSITE

The specified crown composite (see example II-1) was centrally embedded in a cylinder made of epoxy resin for the measurements according to the thermal hardening described above in the form of a cylinder such that the even surfaces of both cylinders formed a plane—see Diagram 1. As a result, the surface of the composite cylinder was sanded with sandpaper (4000 grit) to achieve a flat surface. The measures specified in the following examples were taken. In the process, the repair composite (see example 11-3) was centrally applied to its flat surface in the form of a cylinder having a smaller diameter than that of the crown composite cylinder. The smaller cylinder was then cut off with a shearing blade; the specified shear strength is the value that was measured prior to the breaking off of the cylinder. The higher the shear strength was, the more often and in a greater scope the cracking of the crown composite material was observed in the process, which demonstrates that the bond between the composites is so powerful that it exceeds the breaking strength of the material itself.

### Example V-1

Surface of the crown composite was blasted with corundum (250 μm grain size) with 2.8 bar of pressure at a vertical distance of 1 cm from the surface; a perforated film was subsequently adhered to achieve a defined diameter of the contact surface; Resin System A with 0.6% CC and 0.9% DABE applied and polymerized for 30 seconds with blue light, with the help of a silicon ring, the cylinder made of repair composite was polymerized in one step for 60 seconds with blue light; 1 day of storage at 40° C., dry and dark; shear strength: 15±1 MPa (⅔ adhesive, ⅓ brittle-ductile transition)

### Example V-2

Surface of the crown composite was blasted with corundum (250 μm grain size) with 2.8 bar of pressure at a vertical distance of 1 cm from the surface, thiol TMPMP (trimethylolpropane-tris(3-mercaptopropionate)+(potentially alkaline catalyst) with 0.6% CC and 0.9% DABE tempered for 2 hours at 60° C., subsequently thiol and sample tempered for 1 hour at 40° C., thiol applied to crown composite, tempered for 30 minutes at 40° C. and polymerized for 30 seconds with blue light, excess thiol was then discharged, the cylinder made of repair composite was polymerized to the crown composite in one step for 100 seconds with blue light; 1 day of storage at 40° C., dry and dark; shear strength: 14±1 MPa (⅙ adhesive, ⅚ brittle-ductile transition)

### Example V-3

Surface of the crown composite was blasted with corundum (110 μm grain size) with 2.8 bar of pressure at a vertical distance of 1 cm from the surface; then Hexamethylene-1,6-diisocyanate is applied, the cylinder made of repair composite was polymerized to the crown composite in one step for 100 seconds with blue light; 1 day of storage at 40° C., dry and dark; shear strength: cohesively broken in the crown composite, bond intact.

### Example V-4

Surface of the crown composite was blasted with corundum (250 μm grain size) with 2.8 bar of pressure at a vertical distance of 1 cm from the surface; then Hexamethylene-1,6-diisocyanate is applied, the cylinder made of repair composite was polymerized to the crown composite in one step for 100 seconds with blue light; 1 day of storage at 40° C., dry and dark; shear strength: cohesively broken in the crown composite, bond intact.

### Example V-5

Surface of the crown composite was blasted with corundum (110 μm grain size) with 2.8 bar of pressure at a vertical distance of 1 cm from the surface; then Hexamethylene-1,6-diisocyanate is applied, activated in a pot filled with argon for 30 min. at 60° C., the cylinder made of repair composite was polymerized to the crown composite in one step for 100 seconds with blue light; 1 day of storage at 40° C., dry and dark; shear strength: cohesively broken in the crown composite, bond intact

### Example V-6

Surface of the crown composite was blasted with corundum (250 μm grain size) with 2.8 bar of pressure at a vertical distance of 1 cm from the surface; then Hexamethylene-1,6-diisocyanate is applied, activated in a pot filled with argon for 30 min. at 60° C., the cylinder made of repair composite was polymerized to the crown composite in one step for 100 seconds with blue light; 1 day of storage at 40° C., dry and dark; shear strength: cohesively broken in the crown composite, bond intact

### Example V-7

Surface of the crown composite was blasted with corundum (250 μm grain size) with 2.8 bar of pressure at a vertical distance of 1 cm from the surface; then bonding agent 1 is applied, the cylinder made of repair composite was polymerized to the crown composite in one step for 100 seconds with blue light; 1 day of storage at 40° C., dry and dark; shear strength: cohesively broken in the crown composite, bond intact

### Example V-8

Surface of the crown composite was blasted with corundum (250 μm grain size) with 2.8 bar of pressure at a vertical distance of 1 cm from the surface; bonding agent subsequently applied pursuant to Example IV-2, the cylinder made of repair composite was polymerized to the crown composite in one step for 100 seconds with blue light; 1 day of storage at 40° C., dry and dark; shear strength: cohesively broken in the crown composite, bond intact

### Example V-9

Surface of the crown composite was blasted with corundum (250 μm grain size) with 2.8 bar of pressure at a vertical distance of 1 cm from the surface; bonding agent subsequently applied pursuant to Example IV-3, the cylinder made of repair composite was polymerized to the crown composite in one step for 100 seconds with blue light; 1 day of storage at 40° C., dry and dark; shear strength: cohesively broken in the crown composite, bond intact.

### VI. EXAMPLES FOR THE MEASUREMENT OF THE BOND OF THE REPAIR COMPOSITE TO THE FILLING COMPOSITE

Said filling composite was hardened for the measurements from two sides for 2 minutes with blue light and subsequently embedded in epoxy resin as described for said crown composite and sanded with sandpaper (4000 grit) to achieve a flat surface.

### Example VI-1

Surface of said filling composite (see example 11-2) with corundum (250 μm grain size) with 2.8 bar of pressure at a vertical distance of 1 cm from the surface, Resin System A applied with 0.6% CC and 0.9% DABE to the filling composite and polymerized for 30 seconds with blue light, with the help of a silicon ring, the cylinder made of repair composite was polymerized to the filling composite in one step for 60 seconds with blue light; 1 day of storage at 40° C., dry and dark; shear strength: 14±1 MPa (⅓ adhesive, ⅔ brittle-ductile transition)

### Example VI-2

Surface of said filling composite with corundum (250 μm grain size) with 2.8 bar of pressure at a vertical distance of 1 cm from the surface, thiol TMPMP (trimethylolpropane-tris(3-mercaptopropionate) (+potentially alkaline catalyst) with 0.6% CC and 0.9% DABE tempered for 2 hours at 60° C., subsequently thiol and sample tempered for 1 hour at 40° C., thiol applied to filling composite, tempered 30 minutes at 40° C. and polymerized with blue light for 30 seconds, excess thiol was then discharged, with the help of a silicon ring, the cylinder made of repair composite was polymerized to the filling composite in one step for 100 seconds with blue light; 1 day of storage at 40° C., dry and dark; shear strength: 13±1 MPa (⅙ adhesive, ⅚ brittle-ductile transition)

### REFERENCE LIST FOR DIAGRAM 1

1 Shearing direction of the applied composite cylinder
2 Bond between the composites
3 Cylinder made of repair composite
4 Crown or filling composite
5 Embedding material (epoxy resin)

What is claimed is:

1. Organically-modified silicic acid (hetero) polycondensate capable of being organically cross-linked or a composite, comprising an organically-modified silicic acid (hetero) polycondensate capable of being organically cross-linked and a filler for intraoral use upon repairing an existing dental replacement in the mouth of a patient or for applying an enamel layer to an anatomically-reduced, milled crown base body already located in the mouth of a patient, wherein said existing dental replacement or said crown base body is comprised of a material having free hydroxy, carboxyl, thio or amino groups, or forming these on its surface following a physical-chemical treatment and/or having activated or non-activated C═C double bonds, comprising the following steps in the mouth of the patient:
(a) If necessary, processing of the previously existing dental replacement to remove damaged or no longer necessary elements,
(b) Roughening the surface of the potentially processed dental replacement or said crown base body and/or applying a bonding agent to the surface of said potentially processed dental replacement or said crown base body,
(c) Overlaying the roughened surface and/or the surface coated with bonding agent of said potentially processed dental replacement or said crown base body with said organically-modified silicic acid (hetero) polycondensate or said composite in a liquid or paste-like form, and
(d) Hardening the overlay achieved pursuant to (c) through organic cross-linking by means of light hardening or redox-induced hardening,
wherein said bonding agent is or has a compound having at least two identical or different substituents.

2-28. (canceled)

29. Method for the modification of the surface of a previously hardened or cured shaped article, comprised of a material having free hydroxy, carboxyl, thio or amino groups, or forming these on its surface following a physical-chemical treatment and/or having activated or non-activated C═C double bonds, comprising the following steps:
(a) If necessary, processing of the previously hardened or cured shaped article to remove damaged or no longer necessary elements,
(b) Roughening the surface of the previously hardened or cured shaped article and/or applying a bonding agent to the surface of the previously hardened or cured shaped article, wherein said bonding agent is or has a compound having at least two identical or different substituents
(c) Overlaying the roughened surface and/or the surface coated with bonding agent of said previously hardened or cured shaped article with an organically-modified silicic acid (hetero) polycondensate capable of being organically cross-linked or with a composite, comprising an organically-modified silicic acid (hetero) polycondensate capable of being organically cross-linked and a filler, in a liquid or paste-like form, and
(d) Hardening the overlay achieved pursuant to (c) through organic cross-linking by means of light hardening or redox-induced hardening,

30. Method according to claim 29,
further comprising at least one of the two following steps:
(e) Grinding out a desired form and
(f) Polishing said form.

31. Method according to claim 29, wherein said previously hardened or cured shaped article is selected amongst materials, which are comprised of a cured, organically-modified silicic acid (hetero) polycondensate or a composite, comprising an organically-modified silicic acid (hetero) polycondensate and a filler or contain this material.

32. Method according to claim 29, wherein with the application pursuant to step (b) either the surface of said previously hardened or cured shaped article is chemically etched and/or said previously hardened or cured shaped article is swollen, whereupon said bonding agent is applied to its surface pursuant to step (b).

33. Method according to claim 29, wherein the method additionally comprises the formation of free OH groups on the surface of said previously hardened or cured shaped article before said bonding agent is applied to its surface.

34. Method according to claim 29, wherein a first of the identical or different substituents of the compound, of which said bonding agent was made or which it comprises, is selected from the group consisting of hydroxy groups, isocyanate groups, epoxy groups, potentially activated carboxylic acid groups, thiol groups, primary and secondary amino groups, cyclical carbonate groups, and groups having a potentially activated C═C double bond, and a second substituent of this compound either likewise selected from this group or from among monoalkoxysilyl, dialkoxysilyl, trialkoxysilyl, and Si-bonded OH, providing that compounds having alkoxysilyl or Si—OH groups are silanes or silicic acid poly (partial) condensates.

35. Method according to claim 34, wherein said bonding agent is selected from among dihydroxy compounds, diisocyanates, dicarboxylic acid, the carboxylic acid groups of which are potentially activated, dithiols, diamines, and bis-epoxies.

36. Method according to claim 34, wherein said bonding agent used in the application is a hydrocarbon potentially interrupted by linkage groups preferably selected from among COO, CONH, NHCO or oxygen or sulfur atoms or NH groups and bearing the specified minimum two identical or different functional groups.

37. Method according to claim 34, wherein said bonding agent used in the application comprises a silane having two identical substituents, wherein said silane either has a radical bonded to silicon via carbon, which bears both of the specified substituents, or said silane has two preferably identical radicals bonded to said silicon via carbon, of which each bears one of the two specified identical substituents, or comprises a disilane, an organic component of which is substituted with at least said two specified identical substituents.

38. Method according to claim 29, wherein the previously hardened or cured shaped article is a dental replacement or crown base body milled to an anatomically reduced shape, wherein after roughening and/or applying said bonding agent pursuant to step (b), the roughened surface of the potentially processed dental replacement or of the crown base body and/or the surface of the potentially processed dental replacement or of the crown base body coated with bonding agent is overlaid with an organically-modified silicic acid (hetero) polycondensate in a liquid or paste-like form, to which a color pigment is added for the adaption of the dental replacement or crown base body to the color of the surrounding teeth before step (c) occurs.

39. Method according to claim 38, wherein the organically-modified silicic acid (hetero) polycondensate provided with a color pigment has the same composition as the organically-modified silicic acid (hetero) polycondensate used in step (c).

40. Method according to claim 29, wherein the previously hardened or cured shaped article is a dental replacement or crown base body milled to an anatomically reduced shape, wherein said bonding agent has a color pigment to adapt the dental replacement or crown base body to the color of the surrounding teeth.

41. Method according to claim 29, the method being intraorally performed, wherein the previously hardened or cured shaped article is an already existing dental replacement or is a crown base body milled to an anatomically reduced shape to which an enamel layer is applied.

42. Method according to claim 29, the method being performed in a dental laboratory, wherein the previously hardened or cured shaped article is an already existing dental replacement or is a crown base body milled to an anatomically reduced shape to which an enamel layer is applied.

43. Method according to claim 29, wherein the dental replacement or material is selected from the group consisting of chairside crowns, inlays, onlays, crowns, basic prosthesis materials, dentures, veneer material or fillings.

44. Method for bonding a previously cured material having free hydroxy, carboxyl, thio or amino groups, or forming these on its surface following a physical-chemical treatment and/or having activated or non-activated C═C double bonds with a material made from an organically-modified, organically-cross-linked silicic acid (hetero) polycondensate or a composite, comprising an organically-modified, organically-cross-linkable silicic acid (hetero) polycondensate and a filler, comprising the following steps:
(a) If necessary, processing of the cured material to remove damaged or no longer necessary elements,
(b) Roughening the surface of the potentially processed cured material and/or applying a bonding agent to the surface of said potentially processed cured material,
(c) Overlaying the roughened surface and/or the surface coated with bonding agent of said potentially processed cured material with said organically-modified silicic acid (hetero) polycondensate or said composite in a liquid or paste-like form, and
(d) Hardening the overlay achieved pursuant to (c) through organic cross-linking,
wherein said bonding agent is or has a compound having at least two identical or different substituents.

45. Method for bonding a previously cured material according to claim 44, further comprising at least one of the two following steps:
(e) Grinding out a desired form and
(f) Polishing said form.

46. Method according to claim 44, wherein a first of the identical or different substituents of the compound, of which said bonding agent was made or which it comprises, is selected from the group consisting of hydroxy groups, isocyanate groups, epoxy groups, potentially activated carboxylic acid groups, thiol groups, primary and secondary amino groups, cyclical carbonate groups, and groups having a potentially activated C═C double bond, and a second substituent of this compound either likewise selected from this group or from among monoalkoxysilyl, dialkoxysilyl, trialkoxysilyl, and Si-bonded OH, providing that compounds having a alkoxysilyl or Si—OH group are silanes or silicic acid poly (partial) condensates.

47. Method according to claim 44, wherein in step (b) either the surface of said potentially cured material is chemically etched and/or said potentially processed, previously cured material is swollen, whereupon said bonding agent is applied to its surface pursuant to step (b).

48. Method according to claim 44, further comprising the formation of free OH groups on the potentially roughened and/or swollen surface of said potentially processed, previously cured material before said bonding agent is applied to its surface.
